(19) United States
(12) Patent Application Publication
(10) Pub. No.: US 2014/0271787 A1
(43) Pub. Date: Sep. 18, 2014

(54) CONTINUOUS SINGLE LAYER FILM STRUCTURE INCLUDING DISCRETE DOMAINS

(71) Applicant: MONOSOL RX, LLC, (US)

(72) Inventors: B. Arlie Bogue, New Carlisle, IN (US); Garry L. Myers, Kingsport, TN (US); Alexander Mark Schobel, Whitehouse Station, NJ (US); Eric Dadey, Furlong, PA (US)

(73) Assignee: MONOSOL RX, LLC, Warren, NJ (US)

(21) Appl. No.: 13/839,372

(22) Filed: Mar. 15, 2013

(51) Int. Cl. A61K 9/70; A61K 47/38; A61K 47/10

(52) U.S. Cl. CPC A61K 9/7007; A61K 47/10; A61K 47/38
USPC 424/443; 514/781; 427/2.31; 264/171.1

(57) ABSTRACT

Disclosed herein is a soft, flexible, continuous, single-layer structural directional active delivery device such as a film product with discrete domains useful for controlled administration of the active to a patient. The discrete domains are substantially inseparable from one another, have different rates of dissolution and advantageously offer superior adhesion to the body of the patient while administering the active directionally and effectively.

### FIELD OF THE INVENTION

This invention discloses a soft, flexible, continuous, single-layer structural active delivery device such as a film with discrete domains useful for controlled, directional administration of the active to a patient.

### BACKGROUND OF THE INVENTION

Film delivery systems provide an improved device to administer agents to a user, such as pharmaceutical agents, bioactive agents, nutraceutical agents, and the like. Film delivery systems allow a user to gain administration of the agent without having to use other methods, such as swallowing pills, injection, or other methods that are uncomfortable to the user. While film delivery is gaining popularity, one drawback of film delivery is that as the polymeric film dissolves, the agent is swallowed by the user, and the agent enters the gastrointestinal system. While this may be acceptable for some agents, there are many agents that will become easily destroyed upon entry into the gastrointestinal system.

For this reason, it may be helpful to provide a film system, which can be adhered to a mucosal surface of the user such as the oral (e.g., buccal, sublingual, lingual, gingival), vaginal, ocular and anal areas of the body, and the agent absorbed through that surface into the bloodstream. Direct delivery through the user's skin surface avoids the destruction caused by ingestion.

To allow for absorption, it is desired to provide a film device that can adhere to the mucosal surface for a desired time period, while still allowing for dissolution of the active-containing polymer at the mucosal surface. There are several multi-layered films known, where the inside layer includes an agent and the outside layer is free of an agent, which provide directional delivery of the agent. Multi-layered films, however, suffer from numerous defects, including those during the processing stage (which typically includes lamination by heat and pressure), and post-processing, where the two layers may become separated or destroyed before use.

U.S. Pat. No. 5,047,244 discloses a multilayered film structure suitable to deliver therapeutic agents.

U.S. Pat. No. 5,137,729 discloses a film structure containing a drug-containing layer hot pressed with an alumina or similar layer for delivering drug.

U.S. Pat. No. 5,849,322 discloses a multilayered film for transmucosally administering a drug.

U.S. Pat. No. 6,159,498,244 discloses a multilayered film structure for application to mucosal surfaces for drug delivery.

U.S. Pat. No. 6,585,997 discloses a multilayered film structure for administration of pharmaceuticals to mucosal surfaces.

U.S. Pat. No. 6,780,504 discloses a multilayered film for use as a transmucosal galenic formulation.

U.S. Pat. No. 8,241,661 discloses a single layer film containing a component with a non-uniform distribution in the thickness direction.

There is currently no known single-layer, continuous film structure for directional delivery of an agent. There is no known single-layer, continuous film structure that includes two distinct domains of polymeric material having different thickness, adherence and/or dissolution properties, where the two domains together form a continuous and unitary film structure.

### SUMMARY OF THE INVENTION

In an aspect, the present invention discloses a single layer film dosage structure suitable to be placed against the body of a patient.

In another aspect, the present invention discloses a film dosage structure comprising a single layer film comprising a plurality of discrete domains.

In yet another aspect, the present invention discloses a film dosage structure containing a first domain and a second domain with said first domain comprising a first polymeric carrier material, and said second domain comprising an active and a second polymeric carrier material.

In yet another aspect, the present invention discloses a film dosage structure containing a first domain and a second domain with said first domain comprising a first polymeric carrier material, and said second domain comprising an active and the same polymeric carrier material.

In yet another aspect, the present invention discloses a film dosage structure containing a first domain and a second domain, wherein said second domain has a smaller surface area than the first domain.

In yet another aspect, the present invention discloses a film dosage structure containing a first domain and a second domain, wherein said second domain is at least partially occluded by said first domain.

In yet another aspect, the present invention discloses a film dosage structure containing a first domain and a second domain wherein said first domain and said second domain are physically substantially inseparable, i.e., not peelable apart.

In yet another aspect, the present invention discloses a film dosage structure containing a first domain and a second domain wherein said first domain and said second domain have different rates of dissolution or disintegration when placed in contact with a surface of the body.

In yet another aspect, the present invention discloses a film dosage structure containing a first domain and a second domain wherein said first domain and said second domain have different rates of dissolution or disintegration when placed in contact with a mucosal surface of the body.

In yet another aspect, the present invention discloses a film dosage structure containing a first domain and a second domain wherein said first domain dissolves or disintegrates at a slower rate than the second domain when placed in contact with a mucosal surface of the body.

In yet another aspect, the present invention discloses a film dosage structure containing a first domain and a second domain wherein surface of said second domain is configured to be placed against the body.

In yet another aspect, the present invention discloses a film dosage structure containing a first domain and a second domain wherein surfaces of both said first domain and said second domain are configured to be placed against the body.

In yet another aspect, the present invention discloses a film dosage structure containing a first domain and a second domain wherein surfaces of both said first domain and said second domain are configured to be placed against and in physical contact with the body.

In yet another aspect, the present invention discloses a film dosage structure containing a first domain and a second domain wherein said first domain has a higher level of adherence (in that it dissolves slower) to a bodily surface than said second domain.

In a further aspect, the present invention discloses a film dosage structure suitable to be placed against the body of a patient, said film dosage structure comprising a single layer film comprising a first domain comprising:

(a) a first polymeric carrier material and having a water content of less than 10% by weight of the domain, and

(b) a second domain comprising an active and a second polymeric carrier material and having a water content less than 10% by weight of the second domain, wherein said second domain has a smaller surface area than the first domain and is at least partially occluded by said first domain, said first domain and said second domain are substantially inseparable, and said first domain and said second domain have different rates of dissolution or disintegration when placed in contact with a mucosal surface of the body, and further wherein a surface of said second domain is configured to be placed against the body.

In a further aspect, the present invention discloses a film dosage structure suitable to be placed against the body of a patient, said film dosage structure comprising a single layer film comprising a first domain comprising:

(a) a first polymeric carrier material and having a water content of less than 10% by weight of the domain, and

(b) a second domain comprising an active and the same polymeric carrier material and having a water content less than 10% by weight of the second domain, wherein said second domain has a smaller surface area than the first domain and is at least partially occluded by said first domain, said first domain and said second domain are substantially inseparable, and said first domain and said second domain have different rates of dissolution or disintegration when placed in contact with a mucosal surface of the body, and further wherein a surface of said second domain is configured to be placed against the body

### BRIEF DESCRIPTION OF THE DRAWINGS

FIG. 1 shows a perspective view of one aspect of the film product of the present invention.

FIG. 2 shows a top view of the aspect of FIG. 1.

FIG. 3 shows a bottom view of the aspect of FIG. 1.

FIG. 4 shows a cross-sectional view of the aspect of FIG. 1 taken across the width.

FIG. 5 shows top views of various aspects of the film product of the present invention.

### DETAILED DESCRIPTION OF THE INVENTION

The present invention relates to methods and apparatuses designed for forming film products, including film products that include at least one active composition.

As used herein, the terms “pharmaceutical”, “medicament”, “drug” and “active” may be used interchangeably, and refer to a substance or composition useful for the prevention or treatment of a condition. The terms may include pharmaceuticals, nutraceuticals, cosmetic agents, biologic agents, bioeffective substances, and the like.

It will be understood that the term “film” includes delivery systems of any thickness, including films and film strips, sheets, discs, wafers, and the like, in any shape, including rectangular, square, or other desired shape. The film may be in the form of a continuous roll of film or may be sized to a desired length and width. The films described herein may be of any desired thickness and size suitable for the intended use. For example, a film of the present invention may be sized such that it may be placed into the oral cavity or any body cavity of the user. Such sizes are typically dosage-sized units. Other films may be sized for application to the skin of the user, i.e., a topical use. For example, some films may have a relatively thin thickness of from about 0.1 to about 10 mils, while others may have a somewhat thicker thickness of from about 10 to about 30 mils. For some films, especially those intended for topical use, the thickness may be even larger, i.e., greater than about 30 mils. The composition in its dried film form maintains a uniform distribution of components through the processing of the film. Films may include a pouch or region of medicament between two films.

The term “domain”, as used herein, refers to a polymer-containing region with its own independent properties such as thickness, dissolution, adhesion and the like, which can coexist in intimate contact with another polymer-containing region which has its own independent properties such as thickness, dissolution, adhesion and the like, in a single continuous unitary film structure, wherein the two polymer-containing regions substantially retain their respective independent properties.

Films formed by the present invention may be suitable for administration to at least one region of the body of the user, such as mucosal regions or regions on or within the body of the user, such as on the surface of internal organs. In some aspects of the invention, the films are intended for oral administration. Desirably, the films are intended for topical administration in the oral cavity, but may be used in any bodily cavity or mucosal surface. As used herein, the term “topical agent” is meant to encompass active agents that are applied to a particular surface area. For example, in one aspect, a topical agent is applied to an area of the skin. In other aspects, the topical agent may also be applied to mucosal areas (or mucosal surfaces) of the body, such as the oral (e.g., buccal, sublingual, lingual, gingival), vaginal, ocular and anal areas of the body. It may be understood that films of the present invention may be capable of being applied to more than one mucosal area of the body simultaneously, such as more than one oral mucosal surface. Examples of more than one surface can include, for example, under tongue—floor of mouth, lingual—hard pallet, and buccal—gingival or cheek In still other aspects, the topical agent is applied to an internal organ or a body surface of the user, such as during surgery, where the agent may be removed or left on or within the body after surgery is complete.

Agents useful in the films of the present invention may include any known active, and include, without limitation, those actives set forth in application Ser. No. 13/084,681, filed Apr. 12, 2011, which is incorporated by reference herein in its entirety Any number of active components or pharmaceutical agents may be included in the domain. Various combinations of active components may be used in the same film product to provide a desired effect. For example, the film may include an active and an antagonist, which may be useful for prevention of abuse of the active. Agents useful in the present invention may include pharmaceuticals, bioactive agents, nutraceuticals, proteins, peptides, and the like. Of particular relevance are anti-emetics (such as, for example, ondansetron, granisetron), insulin, and opiates/opiate derivatives (such as, for example, buprenorphine, naloxone). Of still particular relevance is insulin, which has traditionally been administered through injection into the body of a user so as to provide insulin directly into the bloodstream, while avoiding destruction through ingestion. Through the present invention, such agents can be delivered directly through the mucosa into the user's system.

The polymer may be water soluble, water swellable, water insoluble, or a combination of one or more either water soluble, water swellable or water insoluble polymers.

Specific examples of useful water soluble polymers include, but are not limited to, polyethylene oxide (PEO), pullulan, hydroxypropylmethyl cellulose (HPMC), hydroxyethyl cellulose (HPC), hydroxypropyl cellulose, polyvinyl pyrrolidone, carboxymethyl cellulose, polyvinyl alcohol, sodium aginate, polyethylene glycol, xanthan gum, tragancanth gum, guar gum, acacia gum, arabic gum, polyacrylic acid, methylmethacrylate copolymer, carboxyvinyl copolymers, starch, gelatin, and combinations thereof. Specific examples of useful water insoluble polymers include, but are not limited to, ethyl cellulose, hydroxypropyl ethyl cellulose, cellulose acetate phthalate, hydroxypropyl methyl cellulose phthalate and combinations thereof.

As used herein the phrase “water soluble polymer” and variants thereof refer to a polymer that is at least partially soluble in water, and desirably fully or predominantly soluble in water, or absorbs water. Polymers that absorb water are often referred to as being water swellable polymers. The materials useful with the present invention may be water soluble or water swellable at room temperature and other temperatures, such as temperatures exceeding room temperature. Moreover, the materials may be water soluble or water swellable at pressures less than atmospheric pressure. Desirably, the water soluble polymers are water soluble or water swellable having at least 20 percent by weight water uptake. Water swellable polymers having a 25 or greater percent by weight water uptake are also useful. Films or dosage forms of the present invention formed from such water soluble polymers are desirably sufficiently water soluble to be dissolvable upon contact with bodily fluids.

Other polymers useful for incorporation into the films of the present invention include biodegradable polymers, copolymers, block polymers and combinations thereof. Among the known useful polymers or polymer classes which meet the above criteria are: poly(glycolic acid) (PGA), poly(lactic acid) (PLA), polydioxanoes, polyoxalates, poly(α-esters), polyanhydrides, polyacetates, polycaprolactones, poly(orthoesters), polyamino acids, polyaminocarbonates, polyurethanes, polycarbonates, polyamides, poly(alkyl cyanoacrylates), and mixtures and copolymers thereof. Additional useful polymers include, stereopolymers of L- and D-lactic acid, copolymers of bis(p-carboxyphenoxy) propane acid and sebacic acid, sebacic acid copolymers, copolymers of caprolactone, poly(lactic acid)/poly(glycolic acid)/polyethyleneglycol copolymers, copolymers of polyurethane and (poly(lactic acid), copolymers of polyurethane and poly(lactic acid), copolymers of α-amino acids, copolymers of α-amino acids and caproic acid, copolymers of α-benzyl glutamate and polyethylene glycol, copolymers of succinate and poly(glycols), polyphosphazene, polyhydroxy-alkanoates and mixtures thereof. Binary and ternary systems are contemplated.

Other specific polymers useful include those marketed under the Medisorb and Biodel trademarks. The Medisorb materials are marketed by the Dupont Company of Wilmington, Del. and are generically identified as a “lactide/glycolide co-polymer” containing “propanoic acid, 2-hydroxy-polymer with hydroxy-polymer with hydroxyacetic acid.” Four such polymers include lactide/glycolide 100 L, believed to be 100% lactide having a melting point within the range of 338°-347° F. (170°-175° C.); lactide/glycolide 100 L, believed to be 100% glycolide having a melting point within the range of 437°-455° F. (225°-235° C.); lactide/glycolide 85/15, believed to be 85% lactide and 15% glycolide with a melting point within the range of 338°-347° F. (170°-175° C.); and lactide/glycolide 50/50, believed to be a copolymer of 50% lactide and 50% glycolide with a melting point within the range of 338°-347° F. (170°-175° C.).

The Biodel materials represent a family of various polyanhydrides which differ chemically.

The polymer plays an important role in affecting the viscosity of the film. Viscosity is one property of a liquid that controls the stability of the active in an emulsion, a colloid or a suspension. Generally the viscosity of the matrix will vary from about 400 cps to about 100,000 cps, preferably from about 800 cps to about 60,000 cps, and most preferably from about 1,000 cps to about 40,000 cps. Desirably, the viscosity of the film-forming matrix will rapidly increase upon initiation of the drying process.

The viscosity may be adjusted based on the selected active depending on the other components within the matrix. For example, if the component is not soluble within the selected solvent, a proper viscosity may be selected to prevent the component from settling which would adversely affect the uniformity of the resulting film. The viscosity may be adjusted in different ways. To increase viscosity of the film matrix, the polymer may be chosen of a higher molecular weight or crosslinkers may be added, such as salts of calcium, sodium and potassium. The viscosity may also be adjusted by adjusting the temperature or by adding a viscosity increasing component. Components that will increase the viscosity or stabilize the emulsion/suspension include higher molecular weight polymers and polysaccharides and gums, which include without limitation, alginate, carrageenan, hydroxypropyl methyl cellulose, locust bean gum, guar gum, xanthan gum, dextran, gum arabic, gellan gum and combinations thereof.

It has also been observed that certain polymers which when used alone would ordinarily require a plasticizer to achieve a flexible film, can be combined without a plasticizer and yet achieve flexible films. For example, HPMC and HPC when used in combination provide a flexible, strong film with the appropriate plasticity and elasticity for manufacturing and storage. No additional plasticizer or polyalcohol is needed for flexibility.

In embodiments, polyethylene oxide (PEO), when used alone or in combination with a hydrophilic cellulosic polymer, achieves flexible, strong films. Additional plasticizers or polyalcohols are not needed for flexibility. Non-limiting examples of suitable cellulosic polymers for combination with PEO include HPC and HPMC. PEO and HPC have essentially no gelation temperature, while HPMC has a gelation temperature of 58-64° C. (Methocel EF available from Dow Chemical Co.). Moreover, these films are sufficiently flexible even when substantially free of organic solvents, which may be removed without compromising film properties. As such, if there is no solvent present, then there is no plasticizer in the films. PEO based films also exhibit good resistance to tearing, little or no curling, and fast dissolution rates when the polymer component contains appropriate levels of PEO.

To achieve the desired film properties, the level and/or molecular weight of PEO in the polymer component may be varied. Modifying the PEO content affects properties such as tear resistance, dissolution rate, and adhesion tendencies. Thus, one method for controlling film properties is to modify the PEO content. For instance, in some embodiments rapid dissolving films are desirable. By modifying the content of the polymer component, the desired dissolution characteristics can be achieved.

In accordance with the present invention, PEO desirably ranges from about 20% to 100% by weight in the polymer component. In some embodiments, the amount of PEO desirably ranges from about 1 mg to about 200 mg. The hydrophilic cellulosic polymer ranges from about 0% to about 80% by weight, or in a ratio of up to about 4:1 with the PEO, and desirably in a ratio of about 1:1.

In some embodiments, it may be desirable to vary the PEO levels to promote certain film properties. To obtain films with high tear resistance and fast dissolution rates, levels of about 50% or greater of PEO in the polymer component are desirable. To achieve adhesion prevention, i.e., preventing the film from adhering to the roof of the mouth, PEO levels of about 20% to 75% are desirable. In some embodiments, however, adhesion to the roof of the mouth may be desired, such as for administration to animals or children. In such cases, higher levels of PEO may be employed. More specifically, structural integrity and dissolution of the film can be controlled such that the film can adhere to mucosa and be readily removed, or adhere more firmly and be difficult to remove, depending on the intended use.

The molecular weight of the PEO may also be varied. High molecular weight PEO, such as about 4 million, may be desired to increase mucoadhesivity of the film. More desirably, the molecular weight may range from about 100,000 to 900,000, more desirably from about 100,000 to 600,000, and most desirably from about 100,000 to 300,000. In some embodiments, it may be desirable to combine high molecular weight (600,000 to 900,000) with low molecular weight (100,000 to 300,000) PEOs in the polymer component.

For instance, certain film properties, such as fast dissolution rates and high tear resistance, may be attained by combining small amounts of high molecular weight PEOs with larger amounts of lower molecular weight PEOs. Desirably, such compositions contain about 60% or greater levels of the lower molecular weight PEO in the PEO-blend polymer component.

To balance the properties of adhesion prevention, fast dissolution rate, and good tear resistance, desirable film compositions may include about 50% to 75% low molecular weight PEO, optionally combined with a small amount of a higher molecular weight PEO, with the remainder of the polymer component containing a hydrophilic cellulosic polymer (HPC or HPMC).

The present invention provides a continuous, unitary film product, which includes at least two discrete domains. The film product includes a first domain, which is generally a polymeric material and encompasses the majority of the film product by size and weight. This first domain may include an active or it may be free of active. The film product includes a second domain, which includes an active, and further also includes a polymeric material. This second domain is sized and shaped to be present in a smaller amount than the first domain, both in terms of physical size, area and weight.

The first domain desirably is occlusive to the second domain, forming a generally perimetric region around the second domain, as will be explained in further detail below. It is particularly desirable that both the first domain and the second domain include at least one exposed surface on a top side of the film product, whereby each domain can be in contact with a bodily surface simultaneously. Further, as will be described in further detail below, the second domain should have a rate of dissolution or disintegration that is higher than that of the first domain. In addition, the first domain may have a higher level of adherence to a bodily surface than the second domain.

Ways of controlling and differentiating the dissolution or disintegration of the two domains are by either differences in intensive physical properties or differences in extensive physical properties. In the physical sciences, an intensive property (also called a bulk property, intensive quantity, intensive variable or intrinsic property), is a physical property of a system that does not depend on the system size or the amount of material in the system: it is scale invariant. By contrast, an extensive property (also extensive quantity, extensive variable, extensive parameter or extrinsic property) is one that is additive for independent, noninteracting subsystems. It is directly proportional to the amount of material in the system.

For this invention, examples of intensive physical properties which can change dissolution or disintegration are the type of polymers, polymer MW, solubility of the polymer system, moisture content, inclusion of disintegrants, annealing, crosslinking, etc. Examples of extensive properties which can change dissolution or disintegration are surface area, thickness, volume, mass, etc.

For example, by selecting the polymer(s) for the individual domains based on appropriate differences in molecular weight, binding moieties (e.g., carboxylic acid groups, carboxylate groups or hydroxyl groups in one polymer versus the other), viscosity of the polymer, thickness of the layer and such similar properties, as well as combination of such properties. Also, for example, the first domain and the second domain may be from polymer solutions that have different viscosities, for example, generally at least 10% differential viscosities, preferably more than 20% difference in viscosities and more preferably more than 30% difference in viscosities.

Also for example, domains can contain the same polymers in the same ratios and the individual domains can reflect differences in dissolution and disintegration by differences in size, thickness and/or surface area.

The film product is a generally continuous, unitary structure. That is, although the film product includes two or more domains, these domains are not separable from each other. As used herein, the term “continuous, unitary structure” refers to a structure that does not include regions or layers that can easily be separated from each other, such as by peeling apart or wedging the regions away from each other. A “continuous, unitary structure”, used herein, includes a product that has two or more domains of polymeric material in which the polymers in a first domain have become at least partially entangled with the polymers in a second domain. The entanglement could be by any suitable means such as, for example, physical intertwining, chemical bonding, complexing, solvent bonding, and such similar methods. One example of a product that is not a “continuous, unitary structure” as defined herein is a multi-layered, laminated structure.

The film product can be configured, for example, as patterns. For example, there can be a pattern of the second domain on the first domain.

The film product can be configured such that, where example, the second domain is substantially centered on the first domain.

The film product can be configured such that, for example, the second domain is off-centered on the first domain.

The film product can be configured such that, more than one active is present in the same domain or in different domains. Thus, for example, the first domain may contain one active agent, while the second domain may contain a different active. The product may be configured to deliver each drug at separate intervals. Alternately, the first domain may contain an active, while the second domain may contain a placebo. Such configurations are also to be considered aspects of the present invention.

With reference to the Figures, a film product of the present invention is now described.

FIGS. 1-4 show one aspect of the present invention. A continuous film product 10 is provided, which is shown as being a generally rectangular film product. It is understood, of course, that the film product may take other shapes, including square, circular, trapezoid, triangular, or other shapes or designs. Similarly, the discrete domains on the product may have the same shape or different shapes. The film product 10 has a first domain 20 and a second domain 30. The film product 10 has a thickness T as measured from the top surface 40 of the film product 10 to the bottom surface 50 of the film product 10. The film product 10 has a length L and a width W, measured across its sides.

As can be seen in the Figures, the first domain 20 provides a majority of the film product 10, while the second domain 30 encompasses a smaller area than the first domain 20. The film product 10 includes a top surface 40, which is intended to be applied against a bodily surface of the user. The second domain 30 is at least partially occluded by the first domain 20, and desirably the occlusion forms a perimetric region 60 around the second domain 30. That is, the second domain 30 is at least partially surrounded by a perimetric region 60 of the first domain 20. The top surface 40 of the film product 10 includes an exposed surface of the first domain 20 and the second domain 30, such that, when the film product 10 is placed against the user's body, both domains 20 and 30 are in contact with the user's body. In one embodiment, the height of the second domain is higher than that of the first domain; the second domain is in first contact with the mucosal tissue. In another embodiment, the height of the second domain is the same as that of the first domain with both domains contacting the tissue simultaneously.

In one aspect, as can be seen in FIG. 4, the thickness T of the film product 10 may vary at different regions of the film product 10. In particular, the thickness T of the film product 10 may be greater at regions in which the second domain 30 is present than in regions in which the second domain 30 is not present. That is, in regions where the film product 10 encompasses only the first domain 20, the thickness T is relatively small, and in regions where the film product 10 encompasses the first domain 20 and the second domain 30, the thickness T is relatively larger. In some aspects, the thickness T of the film product 10 is substantially uniform throughout the film product. The thickness T of the resulting film product 10 at the region where the second domain 30 is not present is from about 5 to about 10 mils, and more desirably from about 6 to about 8 mils. In one embodiment, the thickness T of the resulting film product 10 at the region where the second domain 30 is present is desirably larger than where 30 is not present, and is desirably from about 8 mils to about 12 mils, and more desirably from about 9 to about 10 mils. The thickness T of the resulting film product 10 at the region where the second domain 30 is not present is desirably about 2 mils to about 6 mils less than the thickness T of the region where the second domain 30 is present. The occlusive domain desirably has a thickness and dissolution rate such that the first domain is slower to dissolve than the second domain.

As discussed above, the first domain 20 desirably forms a substantially perimetric region 60 around the second domain 30. The size of the perimetric region 60 may be as great or small as desired. The size of the perimetric region 60 as measured from the second domain 30 to the edge of the film product 10 is about 1 mm to about 6 mm, and more desirably from about 2 mm to about 4 mm. The perimetric region 60 may be greater along the length L of the film product 10, or it may be greater along the width W of the film product 10.

The first domain 20 is a substantially bioadhesive domain, which is capable of adhering to a bodily surface, such as the mucosal surfaces described above. The terms “bioadhesion” and “bioadhesive” in the present context generally refer to the attachment of synthetic or natural polymers to a biological substrate, as defined by Robinson, J R, “Rationale of Bioadhesion/mucoadhesion”, in Gurny R. Junginger H. E. eds. Bioadhesion: Possibilities and Future Trends, Stuttgart: Wissenschaftliche Verlagsesellschaft, Stuttgart, pages Vol. 13 page 15 (1990) If the substrate is mucus or a mucus membrane, it is also called mucoadhesion. If the substrate is a buccal membrane, it is also called bucoadhesive. It is particularly desirable that the first domain be capable of adhering to various regions of the oral cavity or other body cavities, including the buccal cavity, for a sufficient period of time to allow delivery of the active.

The first domain 20 desirably has a rate of dissolution or disintegration that is slower than that of the second domain 30. In some aspects, the first domain 20 may have a dissolution or disintegration time, when in contact with a bodily surface, such as, for example, a mucosal surface, of at least 5 minutes, at least 10 minutes, at least 15 minutes, or at least 20 minutes.

The second domain 30 desirably has a rate of dissolution or disintegration, when in contact with a bodily surface, such as, for example, a mucosal surface, that is faster than that of the first domain 20. In some aspects, the second domain 30 may have a dissolution or disintegration time of less than 5 minutes, less than 2 minutes, less than 1 minute, less than 30 seconds, or less than 10 seconds.

The rates of dissolution or disintegration of the first domain 20 and the second domain 30 may be selected with consideration to each of their respective rates. That is, the particular rate of dissolution or disintegration of the first domain 20 may be selected so as to provide sufficient time for the second domain 30 to be dissolved or disintegrated. In some aspects, the rate of dissolution or disintegration of the first domain 20 may be substantially the same as that of the second domain 30. In some other aspects, the rate of dissolution or disintegration of the first domain 20 may be about two to about one hundred times the rate of dissolution or disintegration of the second domain 30. Thus, if the rate of dissolution of the second domain 30 is about 10 seconds, the rate of dissolution of the first domain 20 may be from about 20 seconds to about 1000 seconds.

In some aspects, the rates of dissolution or disintegration of the first domain 20, when in contact with a bodily surface, such as, for example, a mucosal surface, may be selected so as to provide a certain time period after dissolution or disintegration of the second domain 30 is substantially complete. For example, it may be desired that the first domain 20 not fully dissolve or disintegrate until about 1 minute to about 30 minutes after the second domain 30 is dissolved or disintegrated. Thus, if the rate of dissolution or disintegration of the second domain 30 is about 30 seconds, then the rate of dissolution or disintegration of the first domain 20 could be from about 1 minute and thirty seconds to about 30 minutes and thirty seconds.

Desirably, the first domain 20 has a rate of dissolution or disintegration of about 1 to about 20 minutes, more desirably from about 2 to about 15 minutes, most desirably from about 5 minutes to about 10 minutes, while the second domain 30 has a rate of dissolution or disintegration of about 10 seconds to about 3 minutes, more desirably from about 20 seconds to about 2 minutes, and most desirably from about 30 seconds to about 1 minute. The first domain 20 will have a rate of dissolution or disintegration that is slower than the second domain 30 by at least one minute and more desirably by at least 5 minutes.

The first domain 20 and the second domain 30 include various film-forming components, including polymers and solvents, in addition to any optional components such as flavors, colors, sweeteners and the like. The polymer or polymers used in each domain may be the same or they may be different. If they are different, the polymers should be compatible with each other to the extent they can adhere to each other or physically intertwine, chemically bond, complex, solvent bond, or interact with each other by such similar methods. In some aspects, the first domain 20 includes polymers selected from the group consisting of polyethylene oxide, cellulose derivatives, and combinations thereof. The polyethylene oxide used in the first domain 20 may have a molecular weight of from about 100,000 to about 900,000. While polymers with higher molecular weights (e.g. up to about 7 million) may be useful, they can be used only at low levels.) In some aspects, the first domain 20 may include polyethylene oxide of higher molecular weight (from about 600,000 to about 900,000) and lower molecular weight (from about 100,000 to about 300,000) in combination. Preferred cellulose derivatives include, for example, methylcellulose and (hydroxypropyl methylcellulose, “HPMC”), and derivatives thereof. Other polymers such as polyvinylpyrrolidinone (“PVP”) as well as combination of PVP and vinyl acetate (“PVP/VA”) may also be used. The polymer(s) in the first domain 20 as well as their molecular weights are desirably chosen so as to provide a desired slower dissolution or disintegration time, as explained above. The differences in the rate of dissolution or disintegration between the first domain and the second domain can also be adjusted by selecting suitable particular solvent(s).

The second domain 30 may include the same polymer or polymers as the first domain 20 or it may include different polymers. The polymer used (e.g. polyethylene oxide) in the second domain 20 may have a molecular weight of from about 100,000 to about 900,000, and more desirably the second domain 20 includes polyethylene oxide of lower molecular weight (from about 100,000 to about 300,000). Preferred cellulose derivatives include, for example, methylcellulose and hydroxypropyl methylcellulose, and derivatives thereof. The polymer(s) in the second domain 30 as well as their molecular weights are desirably chosen so as to provide a desired faster dissolution or disintegration time, when placed in contact with a bodily surface, such as, for example, a mucosal surface, than the first domain, as explained above.

Each domain may include any amount of film-forming polymers, and in some aspects, each domain may include from about 10% to about 75% polymers, as measured by total weight of the film-forming material in their respective domains prior to any drying steps.

In addition to film-forming polymer(s), each domain may include a solvent or solvents. The solvent or solvents in each domain may be the same or they may be different. Useful solvents include, but are not limited to, polar solvents such as water. Non-polar solvents may be used if desired. Combinations of polar and non-polar solvents are also useful. In addition, the amount of solvent or solvents in each domain may be the same or they may be different. Non-limiting examples of useful solvents may include water, ethanol, propanol, acetone and combinations thereof. During the formation of the product, that is, prior to any drying steps, there may be at least 15% solvent by weight of the film-forming material. In some aspects, more solvent may be used, and thus each domain may have up to around 70% by weight solvent.

Other materials may be included in the film-forming materials, either in the first domain 20, second domain 30, or both domains. Such materials may include sweeteners, flavors, colors, fillers, and the like. For example, one or both domains may include sugar or sugar-free sweeteners, such as polyols.

One or both domains may additionally include an active material, as described above. The active material may be found in one or both domains, and more than one active material may be used in either domain or both domains. In some aspects, the first domain 20 includes a first active and the second domain 30 includes a second active, where the first active and second active may be the same or they may be different. It is particularly desirable that the second domain 30 include an active material which is to be absorbed by the bodily surface to which the product 10 is adhered, which will be explained in further detail below.

In preferred aspects, the dissolution times of the first domain 20 and the second domain 30 are different, as explained above. The choice of polymers and their respective molecular weights in each of the domains plays an important role in determining the dissolution or disintegration times of the particular domains. Higher molecular weight polymers, for example, have a tendency to slow the rate of dissolution or disintegration, and may be more useful in the first domain 20 than in the second domain 30. Other physical modifications may be used to modify the dissolution or disintegration times of each domain, including, for example, modifying the amount or type of solvent in each of the domains. In systems where the domains contain the same polymers or polymer ratios, the use of extensive properties such as thickness, mass or surface area can be used to modify dissolution or disintegration times. It is particularly desired that the domains have related dissolution or disintegration times, as explained in more detail above.

The product 10 desired is a unitary, continuous structure of at least two domains, such as first domain 20 and second domain 30. The unitary structure of the product 10 may be achieved through physical intertwining of polymers in the two domains, or may be achieved through solvent bonding, chemical bonding, complexation, ionic bonding, Van der walls forces, hydrogen bonding etc between the domains, in other words not physically separable without destruction of the film. Again, it is noteworthy that the product 10 is formed as a unitary structure of domains, which is different from a product formed via lamination.

Lamination of layers creates a number of problems, including manufacturing inefficiencies and higher costs in creating separate layers and laminating them, exposure to excess heat and pressure during formation, separation of layers after formation, and a difficulty achieving an “occluded” area, which is formed by the perimetric region 60 of the first domain 20.

In use, the resulting unitary film product 10 may be placed against a bodily surface, including mucosal surfaces. In use, the top surface 40 of the product 10 is substantially in contact with a bodily surface, such that the top surface 40 of the first domain 20 and the second domain 30 are in contact with a bodily surface at the same time. The perimetric region 60, being formed by the first domain 20, is a substantially adherent material, which will adhere to the bodily surface, keeping the product 10 in place. Desirably, the second domain 30 is made of a material that dissolves too quickly to substantially adhere to the bodily surface, and/or is formed from polymers which do not have bioadhesive properties. Some other ways of controlling and differentiating the two domains are, for example, selecting intensive properties such as the polymer(s) for the individual domain layers based on differences in molecular weight, binding moieties (e.g., carboxylic acid groups, carboxylate groups or hydroxyl groups in one polymer versus the other), viscosity of the polymer or extensive properties such as thickness of the layer, and such similar properties, as well as combination of such properties.

In use, the product 10 is placed against the bodily surface in such a fashion that the top surface 40 is in contact with the bodily surface. If desired, the bodily surface may be wetted prior to adhering the product 10 thereto. The perimetric region 60 of the product 10 adheres to the bodily surface, keeping the product 10 in place. Since the perimetric region 60 of the product 10 is adhered to the bodily surface, the second domain 30 is trapped or occluded by the adhered region 60. Further, as the second domain 30 is occluded by the first domain 20, the second domain 30 is directed towards the bodily surface to which it is adhered, allowing the materials of the second domain 30 to be directionally delivered and controllably absorbed into the bodily surface as desired. Since the second domain 30 has a faster dissolution or disintegration rate than the first domain 20, the active in the second domain 30 can be substantially absorbed by the bodily surface before the first domain 20 is dissolved or disintegrated. Given the controlled delivery of the active of the second domain 30, the use of the present product 10 provides quick, accurate and directional delivery of materials to a user without risk of losing active materials.

The thickness T of the product 10 may vary in regions. As explained above, the thickness T may be greater at a region where the second domain 30 is located than where the second domain 30 is not located. Having a product with a larger thickness T where the second domain 30 is located allows for assurance that the second domain is likely to contact the bodily surface during use, and begin dissolution/disintegration and delivery quickly. Additionally, the different thicknesses can be used to control the dissolution or disintegration rates of the different domains.

The controlled delivery of active contained within the second domain 30 allows for a safe and secure means of administering an active, while at the same time avoiding loss of active through unintentional ingestion of the active. For example, if the product 10 did not include a perimetric region 60 adhered to the bodily surface, there would be a channel or pocket between the product 10 and the bodily surface, through which active may escape during dissolution of the second region 30. Trapping and occluding the second domain 30 against the bodily surface allows for controlled delivery of the active contained therein or thereon.

The present invention also includes a method for making the film product of the present invention. Various methods of forming uniform films, as well as various polymers, additives and fillers, are described in U.S. Pat. Nos. 7,425,292, 7,357,891, 7,666,337, 7,824,588 and 7,897,080, which are herein incorporated by reference in their entireties. The present inventors have discovered that, in order to form a unitary structure having two physically inseparable domains, certain criteria must be met. For example, it is important that any solvent used in one domain (e.g., the first domain) not be allowed to overly and undesirably dissolve the polymer or polymers in the other domain (e.g. the second domain) during processing. During processing, the first domain may be formed and dried to provide a substantially dried film product, and then the second domain applied thereon. To achieve a single, unitary structure, it is useful that the second domain be allowed to at least partially dissolve (or soften) the polymer(s) of the first domain, thereby physically entangling the polymers in both domains and/or chemically bonding or complexing the two domains together. For example, if there are hydrophilic moieties in the second domain they may, upon deposition and diffusion into the first domain, form hydrate or gel-like structure or micelle-like structure, keeping the second domain in place. However, over-dissolution of the first domain by the solvent(s) of the second domain may undesirably destroy the integrity of the first domain, rendering the resulting product useless.

The present inventors have discovered that avoidance of over-dissolution of the first domain may be achieved through a number of different processes. One such process is described herein.

In one particular method of forming the desired film product 10, a film structure is first formed, which will form the first domain 20. A film-forming material or matrix is first formed, using polymers, solvents, fillers, and the like as desired, to provide a film product having the desired dissolution or disintegration time. In some aspects, this first domain 20 may be formed through the use of a combination of polymers, such as polyethylene oxide and cellulose, such as hydroxypropylmethylcellulose (“HPMC”). A film-forming matrix is deposited onto a substrate and then dried to form a film product. Drying may be achieved through any desired suitable method.

A second film-forming material, which will be used to form the second domain, will also be prepared. Desirably, this second material includes an active, and will be used to form a film product having the desired dissolution or disintegration rates described above. In preferred aspects, the dissolution or disintegration rate of the second material (and subsequently second domain) is faster than that of the first material (and subsequently first domain). It may be desired that this second domain be formed through the use of a high solids content film-forming material. By “high solids content”, it is intended that the film-forming matrix include at least 25% solid components, preferably at least 30% solid components, more desirably at least 35% solid components, and most desirably above 50% solid components. Although not critical, it is desired that the solvent used in the first film-forming material and the solvent in the second film-forming material be the same.

In this method, the second film-forming material is deposited onto the dried film product, for example, onto selected areas (discontinuous discrete domains), on a surface of said first dried film to form a wet multi-domain film and then the resulting multi-domain product be dried. It is particularly desired that the deposition and drying process be conducted in such a fashion that the two domains become associated with each other sufficiently that they form a single unitary structure, but still leaving the perimetric region around the second domain. The Applicants have found that, by depositing the second film-forming material onto the dried film product at a location very close to the drying oven, and then exposing the multi-domain product to an initial high heat quickly so as to flash off the solvent, the useful product can be formed.

For example, the second film-forming material may be deposited onto the dried film product at a location that is less than about 1 meter away from the drying oven, or less than about 0.5 meters away from the drying oven, where it travels at a rate of about 0.1 to about 40.0 meters per minute into the oven, preferably from about 0.5 to about 30, more preferably from about 1.0 to about 20, even more preferably from about 2.0 to about 10; and even more preferably from about 0.5 to about 5.0 meters per minute. Once in the oven, the product is dried at an initial temperature of about 30° C. to about 140° C., which allows the solvent in the second film-forming material to be flashed off. In this aspect, the two domains are in contact with each other for sufficient time to allow a slight degree of solvent migration between the second film-forming material and the dried film product, but not too much solvent migration that the dried film product is dissolved.

In an alternative method, the first film-forming material and the second film-forming material may use different solvents or solvent systems. In particular, the solvent used in the first film-forming material will not dissolve the polymer of the second film-forming material, and the solvent used in the second film-forming material will not dissolve the polymer of the first film-forming material. In this alternative aspect, the first film-forming material is deposited and dried to form a first dried film, and then the second film-forming material is deposited onto the first dried film. Since the solvent of the second film-forming material does not dissolve the polymers of the first dried film, the rapid initial drying step described above is not required. For example, it may be desired that the second film-forming material include an ethanol-based material, and the first film-forming material include a water-based material. It is particularly desired that the solvent of the second film-forming material be capable of softening the polymers of the first dried film product, but not dissolve the polymers, e.g. ethanol with small amounts of water. The polymers of the two domains may then be intertwined and thus physically inseparable.

It is important to note that the aforementioned methods are different from typical lamination steps in a number of different ways. First, lamination steps require the use of heat and pressure to achieve results which may have a harmful effect on the materials used. In addition, lamination often results in a multi-layered product that has distinguishable and separable regions which is different from discontinuous discrete domains on a single layer film. Additionally, it is very difficult to produce an occlusive film using melt lamination and to have active domain totally inclusive within the confines of the occlusive domain.

While the above description discusses one method of film deposition, various other methods of film deposition are also contemplated and should be considered as aspects of the present invention. For example, depositions from powders, solids, liquids, granules, beads, pellets, pastes, solutions, suspensions, semi-solids, thixotropes, emulsions and the like are also useful for film depositions in the present invention. Similarly, techniques such as, for example, spray coating, strip coating, patch coating and the like, are also contemplated to be applicable in the practice of the present invention.

### EXAMPLES

### Example 1

### Single Layer Delivery Device

A single layer delivery device was formed with a first and second domain. The first domain included polyethylene oxide, sold under the trade name Polyox WSR-N80 PEO, LEO NF, maltitol syrup lycasin (80/55), glycerin, methyl cellulose sold under the trade name Methocel E15 LV, polyethylene oxide sold under the trade name Polyox WSR-1105 PEO, LEO NF, sucralose, Peceol, coloring agents and water.

A flowable mixture was first formed as follows. The water was first added and agitated at 60 Hz. The liquid components are then added to the water, and the solution was mixed at 60 Hz for about 5 minutes. The powdered components were then added via vacuum addition, and the mixture was then mixed at 60 Hz for about 30 minutes. The solution was degassed during mixing.

Once mixed, the flowable mixture was cast onto a Mylar substrate and dried. The resulting dried first domain had a resulting moisture content of about 8%. The dried first domain was stored on a roll for later use.

The second domain was formed and included polyethylene oxide sold under the trade name Polyox WSR-N10 PEO, LEO, methyl cellulose sold under the trade name Methocel E15 LV, glycerin, maltitol syrup lycasin 80/55, titanium dioxide, Peceol and water. The second domain was formed by agitating water at 60 Hz, and then adding the titanium dioxide, glycerin and Peceol. The resulting mixture was mixed for 5 minutes at 60 Hz. The polyethylene oxide and methyl cellulose were then added and mixed for 30 minutes at 60 Hz. Finally, the maltitol syrup lycasin was added, and the mixture was mixed at 60 Hz for 15 minutes. The solution was degassed during mixing. The resulting wet mixture had a water content of >10%.

The second domain was deposited onto the dried first domain via slot die coating. The second domain was deposited in discrete regions measuring about 25×18 mm. It was dried at about 110° C. for approximately 5 minutes.

The final structure was cut into pieces that measured about 33×22 mm, where the second domain was inclusive within the first domain with edges of about 4 mm on two of the sides and about 2 mm on the other two sides. The thickness of the region of the film product where the active was present was about 9.5-10 mils (241.2-254 microns) and the thickness of the region of the film product where no active was present was about 6.5-7 mils (165-178 microns).

The film components were subjected to a partial immersion dissolution (PID) test, which measures the dissolution of an object in a solvent under stress. The PID test measures dissolution times of a film. The object to be tested was placed in a clamp and inserted into a beaker containing water at a temperature of 37.2 C. The second domain dissolved in about 3 seconds, while the first domain dissolved in about 18-20 seconds. This test correlates to an in vivo dissolution time of less than 10 seconds for the second domain and greater than 3 minutes for the first domain.

### Example 2

### Film Product with Naloxone (an Opioid Antagonist Drug Available Commercially as Nalone®)

The two domains of Example 1 were made according to the same process as Example 1, but the second domain included 0.5 mg of naloxone.

The resulting unitary film product was placed in a convertible glass covered flow cell connected to a pump, which pumps at a rate of 8.89 grams per minute. Distilled water was used as the solvent. The experiment was started and a collection was made at 5, 10, 15, 20, 25, 30, 35 and 40 minute intervals. The results of each collection is set forth in the Table below:

| | | | | Naloxone |
|---|---|---|---|---|
| | Time of | Weight of | | total per |
| Sample No. | Collection | Collection | Assay μg/ml | collection |
| 1 | 5 | 45.07 | 2.386 | 107.5 |
| 2 | 10 | 44.32 | 1.683 | 74.6 |
| 3 | 15 | 44.94 | 1.515 | 68.1 |
| 4 | 20 | 45.09 | 1.448 | 65.3 |
| 5 | 25 | 44.80 | 1.261 | 56.5 |
| 6 | 30 | 44.76 | 1.051 | 47.0 |
| 7 | 35 | 44.75 | 0.851 | 38.1 |
| 8 | 40 | 44.88 | 0.587 | 26.3 |
| | Total: | 358.61 | | 483.4 |

The total assay of naloxone 483.4 micrograms is within the FDA guidelines of +/−10% of the expected target assay (0.5 mg). This experiment indicates that the second domain containing the naloxone dissolved at a faster rate than the first domain, which lasted at least 40 minutes. The active component delivered over 83% of the active naloxone within 30 minutes. This data shows that the concentration drops faster in the first 15 minutes than predicted and slower after that until total disintegration time of the film at 40 minutes.

### Example 3

### Co-Extruded Gold Nanoparticle (GNP) Film

The following solutions were mixed at 40% solids.

| Ingredient (kg) | WT % of Solution | Function |
|---|---|---|
| PEO WSR N10 LEO | 18.69% | Film Former |
| HPMC E15 | 9.35% | Film Former |
| Maltitol Added as Lycasin 80/55* | 6.23% | Plasticizer |
| Glycerin | 4.67% | Plasticizer |
| Sucralose | 0.80% | Sweetener |
| Peppermint 2303 | 1.60% | Flavor |
| Peceol | 0.20% | Defoamer |
| FD & C Blue # 1 Granular | 0.02% | Colorant |
| Water | 58.44% | Solvent |
| total | 100.00% | |

*Maltitol Added as Lycasin 80/55 is added as a 75% (w/w solids) solution.

A second solution was mixed containing 30% solids:

| Ingredient (kg) | WT % of Solution | Function |
|---|---|---|
| PEO WSR N10 LEO | 14.02% | Film Former |
| HPMC E15 | 7.01% | Film Former |
| Maltitol Added as Lycasin 80/55* | 4.67% | Plasticizer |
| Glycerin | 3.50% | Plasticizer |
| Sucralose | 0.60% | Sweetener |
| Peppermint 2303 | 1.20% | Flavor |
| Peceol | 0.15% | Defoamer |
| FD & C Blue # 1 Granular | 0.01% | Colorant |
| Water | 68.83% | Solvent |
| total | 100.00% | |

*Maltitol Added as Lycasin 80/55 is added as a 75% (w/w solids) solution.

These solutions were mixed using the following process:

The ingredients were added in the following order while mixing:

Water

Maltitol Lycasin Syrup 80/55, NF

Glycerin

Sucralose

Peceol, USP

FD&C Blue #1 Granular

Methocel E15, LV, USP

Sentry Polyox WSR N10 LEO, NF

Peppermint 2303

A second solution containing 40% solids with the same solvent, polymer mix and gold nanoparticles was created using the same process. The ingredients were:

| Ingredient | WT % of Solution | Function |
|---|---|---|
| PEO WSR N10 LEO | 19.90% | Film Former |
| HPMC E15 | 9.95% | Film Former |
| Maltitol Added as Lycasin 80/55 * | 6.63% | Plasticizer |
| Glycerin | 4.97% | Plasticizer |
| Peceol | 0.19% | Defoamer |
| Gold Nanoparticle | 1.50% | Active |
| (GNP) solution | | |
| Water | 56.84% | Solvent |
| total | 100.00% | |

By using a duel slot die coating head, a single layer film with a homogenous polymer content ratio was created by the following methods. The non-GNP film was formed with a 180 mm wide single slot die. The GNP film was formed using a multiple slot die, containing 8 slots, each 14 mm wide and 8 mm apart. The resulting film consisted of 8 lanes, each 22 mm wide and containing a 14 mm stripe in the middle of the lane. The dies could be inverted, putting either one on the top. Using the combination of 30% and 40% solids solutions, examples 3a-3g were formed as defined in the following table.

| | Non-GNP | | GNP | |
|---|---|---|---|---|
| | containing solution | | containing solution | |
| | % Solids | | % Solids | |
| Example # | (30%/40%) | Top/Bottom | (30%/40%) | Top/Bottom |
| 3a | 40% | Bottom | 30% | Top |
| 3b | 40% | Bottom | 40% | Top |
| 3c | 30% | Bottom | 30% | Top |
| 3d | 40% | Top | 30% | Bottom |
| 3e | 40% | Top | 40% | Bottom |
| 3f | 30% | Top | 30% | Bottom |
| 3g | 30% | Top | 40% | Bottom |

From sample 3b, film strips were die cut at 22 mm×19.5 mm, weighed and analyzed for gold content. The results are in the following table:

| | |
|---|---|
| Gold | |
| Concentration | Strip Weight |
| (mcg) | (mg) |

| | Average | RSD | Average | RSD |
|---|---|---|---|---|
| Lane 1 | 3.78 | 0.8% | 0.1226 | 0.5% |
| Lane 4 | 3.62 | 2.2% | 0.1193 | 0.4% |
| Lane 8 | 3.54 | 0.6% | 0.1201 | 2.9% |
| Beginning | 3.63 | 2.9% | 0.1211 | 1.8% |
| Middle | 3.68 | 4.0% | 0.1192 | 2.4% |
| End | 3.63 | 3.5% | 0.1217 | 1.5% |
| All | 3.65 | 3.1% | 0.12 | 1.9% |

To evaluate uniformity of content, the % difference was calculated between the minimum values and maximum values for gold concentration and weight ([Max−Min]/average). This was repeated for the % difference between the minimum value and the average value and the maximum value and the average value.

| % | Gold Conc. | Strip Wt |
|---|---|---|
| Difference | (mcg) | (g) |
| Mn | 3.52 | 0.12 |
| Max | 3.81 | 0.12 |
| Ave | 3.65 | 0.12 |
| Max-Min | 8.0% | 5.7% |
| Max-Ave | 4.45% | 1.92% |
| Ave-Min | 3.50% | 3.79% |

The Acceptance Value (AV) for content uniformity was also calculated from the formula: AV=2.4×SD, where the mean value is the target.

In this case, AV=3.1%×2.4=7.44%, where the limit is 15%, so the values meet the CU requirement for pharmaceutical product release.

### Example 4

### Independently Extruded GNP Film

Using the solutions from Example 3, experiments were run where either the non-GNP solution was coated and dried, followed by a second coating of GNP solution or the reverse. GNP solution was coated either as a stripe or patch. The experiments are outlined in the following table:

| | Non-GNP | | GNP | | |
|---|---|---|---|---|---|
| | containing solution | | containing solution | | |
| | % Solids | | % Solids | | Patch |
| Experiment | (30%/ | Top/Bottom | (30%/ | Top/Bottom | or |
| # | 40%) | Wet/Dry | 40%) | Wet/Dry | Stripe |
| 4a | 40% | Bottom/Dry | 30% | Top/Wet | Patch |
| 4b | 40% | Bottom/Dry | 40% | Top/Wet | Patch |
| 4c | 40% | Bottom/Dry | 30% | Top/Wet | Stripe |
| 4d | 40% | Bottom/Dry | 40% | Top/Wet | Stripe |
| 4e | 30% | Top/Wet | 40% | Bottom/Dry | Stripe |
| 4f | 40% | Top/Wet | 40% | Bottom/Dry | Stripe |

### Example 5

### Simultaneously Extruded GNP Film Patches

Using the solutions from Example 3, solution containing GNPs was coated as a patch, approximately 14 mm×18 mm simultaneously with the non-GNP film. These experiments are outlined in the following table.

| | Non-GNP | | GNP | |
|---|---|---|---|---|
| | containing solution | | containing solution | |
| | % Solids | | % Solids | |
| Experiment # | (30%/40%) | Top/Bottom | (30%/40%) | Top/Bottom |
| 5a | 40% | Bottom | 40% | Top |
| 5b | 40% | Top | 40% | Bottom |

### Example 6

### Co-Extruded Solutions with the Same Dimensions

Using the solutions from Example 3, the following examples were formed:

| | Non-GNP | GNP |
|---|---|---|
| | containing | containing |
| | solution | solution |
| | % Solids | % Solids |
| | (30%/ | (30%/ |
| Experiment # | 40%) | 40%) |

Dual 180 mm slot (top/bottom)

| | | |
|---|---|---|
| 6a | 30% | 30% |
| 6b | 40% | 30% |
| 6c | 40% | 40% |

Alternating 12.5 mm slots (side by side)

| | | |
|---|---|---|
| 6d | 30% | 30% |
| 6e | 40% | 30% |
| 6f | 40% | 40% |

The results of this example showed that no matter if the domains were formed one on top of the other or side by side, they still formed one continuous film strip that could not be separated and had a consistent polymer ratio throughout.

It is remarkable that by suitable selection of polymer(s), and/or molecular weight(s) and/or deposition method(s) and/or extensive properties, the present invention yields a single unitary film structure with more than one domain as discontinuous discrete regions, for efficiently and directionally administering an active to a desired location on a patient's body. Such a structure may be efficiently and economically manufactured and the resultant drug delivery properties are also efficient. With such a combination of benefits, the present invention offers substantial advantages over any method known in the art for a film-based drug delivery system.

What is claimed is:

1. A film dosage structure suitable to be placed against the body of a patient, said film dosage structure being a single layer structure comprising:
(a) a first domain comprising a first polymeric carrier material, and
(b) a second domain comprising an active and a second polymeric carrier material, wherein said second domain has a smaller surface area than the first domain and is at least partially occluded by said first domain, said first domain and said second domain are physically substantially inseparable, and said first domain and said second domain have different rates of dissolution or disintegration when placed in contact with a mucosal surface of the body.

2. The film dosage structure of claim 1, wherein both the first and second domains are configured to be in contact with the body.

3. The film dosage structure of claim 1 being a continuous structure.

4. The film dosage structure of claim 1, wherein the polymeric carrier of the second domain dissolves faster, when placed in contact with a bodily surface or a mucosal surface, than the polymeric carrier of the first domain.

5. The film dosage structure of claim 1, wherein the first domain adheres to mucosal tissue.

6. The film dosage structure of claim 1, wherein the second domain adheres to mucosal tissue.

7. The film dosage structure of claim 1, wherein the second domain is substantially non-adherent to mucosal tissue.

8. The film dosage structure of claim 1, wherein second domain is perimetrically surrounded by the first domain.

9. The film dosage structure of claim 1, wherein the first and second domains are con-terminus.

9. The film dosage structure of claim 1, wherein the first polymeric material comprises high molecular weight polyethylene oxide, low molecular weight polyethylene oxide, cellulose derivatives, and combinations thereof.

11. The film dosage structure of claim 1, wherein the second polymeric material comprises high molecular weight polyethylene oxide, low molecular weight polyethylene oxide, cellulose derivatives, and combinations thereof.

12. The film dosage structure of claim 1, wherein the first polymeric material and the second polymeric material are the same.

13. The film dosage structure of claim 1, wherein the first polymeric material and the second polymeric material are different.

14. The film dosage structure of claim 1, wherein the thickness of the structure varies from a region where the second domain is not present to a region where the second domain is present.

15. The film dosage structure of claim 1, wherein the thickness of the film dosage structure is greater at a region where the second domain is present than at a region where the second domain is not present.

16. The film dosage structure of claim 1, wherein both the first domain and the second domain are of the same shape.

17. The film dosage structure of claim 1, wherein the first domain and the second domain are of different shapes.

18. The film dosage structure of claim 16, wherein both the first domain and the second domain are generally circular in shape.

19. The film dosage structure of claim 1, wherein the first domain dissolves or disintegrates at a slower rate than the second domain.

20. The film dosage structure of claim 19, wherein the rates of disintegration, when placed in contact with a bodily surface or a mucosal surface, are about 2 to about 100 times different for the first and second domains.

21. The film dosage of claim 12, wherein said first polymeric material and said second polymeric material are of different molecular weights.

22. The film dosage of claim 21, wherein the molecular weight of said first polymeric material is higher than the molecular weight of said second polymeric material.

23. The film dosage structure of claim 12, wherein both first domain and the second domain comprise polyethylene oxide and cellulose.

24. The film dosage structure of claim 1, wherein said second domain is substantially centered on said first domain.

25. The film dosage structure of claim 1, wherein said second domain is off-centered on said first domain.

26. The film dosage structure of claim 1, wherein said first domain and said second domain are configured to be in contact with a mucosal surface of the body.

27. The film dosage structure of claim 1, wherein there is a pattern of said second domain present on said first domain.

28. A method of forming a continuous and uniform single layer film product comprising a first domain and second domain, wherein said first and second domains are substantially inseparable, comprising the steps of:
(a) preparing a first wet film-forming matrix comprising a first solvent and a first polymeric material;
(b) forming a first wet film by casting said first film-forming matrix;
(c) preparing a second wet film-forming matrix comprising an active, a second solvent and a second polymeric material;
(d) depositing a predetermined amount of said second wet film-forming matrix onto selected areas on a surface of said first wet film to form a wet multi-domain film comprising a first domain and a second domain; and
(e) rapidly feeding said wet multi-domain product into a drying apparatus and exposing said wet multi-domain film to a temperature sufficient to flash off said first and second solvents so as to form a multi-domain product.

29. The method of claim 28, wherein step (e) comprises passing said wet multi-domain film at a rate of about 0.5 to about 5.0 meters per minute through said drying apparatus at an initial temperature of about 30° C. to about 140° C.

30. The method of claim 28, wherein said first polymeric material and said second polymeric material are intertwined.

31. The method of claim 28, wherein the first domain of said multi-domain product adheres to mucosal tissue.

32. The method of claim 28, wherein the second domain of said multi-domain product adheres to mucosal tissue.

33. The method of claim 28, wherein the second domain of said multi-domain product is substantially non-adherent to mucosal tissue.

34. The method of claim 28, wherein the first polymeric material comprises high molecular weight polyethylene oxide, low molecular weight polyethylene oxide, cellulose derivatives, and combinations thereof.

35. The method of claim 28, wherein the second polymeric material comprises high molecular weight polyethylene oxide, low molecular weight polyethylene oxide, cellulose derivatives, and combinations thereof.

36. The method of claim 28, wherein the first polymeric material and the second polymeric material are the same.

37. The method of claim 28, wherein said first polymeric material and said second polymeric material are of different molecular weights.

38. The method of claim 34, wherein the molecular weight of said first polymeric material is higher than the molecular weight of said second polymeric material.

39. The method of claim 36, wherein the first polymeric material and the second polymeric material comprise polyethylene oxide and cellulose.

40. The method of claim 28, wherein the first solvent and the second solvent are the same.

41. The method of claim 28, wherein the first solvent is a water-based solvent.

42. The method of claim 28, wherein the second solvent is a water-based solvent.

43. The method of claim 28, wherein the second solvent is a non-aqueous solvent.

44. A method of forming a continuous and uniform single layer film product comprising a first domain and second domain, wherein said first and second domains are substantially inseparable, comprising the steps of:
(a) preparing a first film-forming matrix comprising a first solvent and a first polymeric material;
(b) preparing a second film-forming matrix comprising an active, a second solvent and a second polymeric material;
(c) co-extruding said first film-forming matrix and said second film-forming matrix to form a wet multi-domain film comprising a first domain and a second domain; and
(e) rapidly feeding said wet multi-domain product into a drying apparatus and exposing said wet multi-domain film to a temperature sufficient to flash off said first and second solvents so as to form a multi-domain product,
wherein said multi-domain product includes a top surface, said top surface comprising a top surface of said first domain and said second domain, wherein said second domain is perimetrically surrounded by said first domain.

45. The method of claim 44, wherein step (e) comprises passing said wet multi-domain film at a rate of about 0.5 to about 5.0 meters per minute through said drying apparatus at an initial temperature of about 30° C. to about 140° C.

46. The method of claim 44, wherein said first polymeric material and said second polymeric material are intertwined.

47. The method of claim 44, wherein the first domain of said multi-domain product adheres to mucosal tissue.

48. The method of claim 44, wherein the second domain of said multi-domain product adheres to mucosal tissue.

49. The method of claim 44, wherein the second domain of said multi-domain product is substantially non-adherent to mucosal tissue.

50. The method of claim 44, wherein the first polymeric material comprises high molecular weight polyethylene oxide, low molecular weight polyethylene oxide, cellulose derivatives, and combinations thereof.

51. The method of claim 44, wherein the second polymeric material comprises high molecular weight polyethylene oxide, low molecular weight polyethylene oxide, cellulose derivatives, and combinations thereof.

52. The method of claim 44, wherein the first polymeric material and the second polymeric material are the same.

53. The method of claim 44, wherein said first polymeric material and said second polymeric material are of different molecular weights.

54. The method of claim 50, wherein the molecular weight of said first polymeric material is higher than the molecular weight of said second polymeric material.

55. The method of claim 52, wherein the first polymeric material and the second polymeric material comprise polyethylene oxide and cellulose.

56. The method of claim 44, wherein the first solvent and the second solvent are the same.

57. The method of claim 44, wherein the first solvent is a water-based solvent.

58. The method of claim 44, wherein the second solvent is a water-based solvent.

59. The method of claim 44, wherein the second solvent is a non-aqueous solvent.
